(19) United States
(12) Patent Application Publication
(10) Pub. No.: US 2016/0326494 A1
(43) Pub. Date: Nov. 10, 2016

(54) Methods for Pancreatic Islet Transplantation

(71) Applicants: Joao Paulo Monteiro Carvalho Mori CUNHA, Lubbeek (BE); Conny Gysemans, Rotselaar (BE); Chantal Mathieu, Buizingen (BE)

(72) Inventors: Joao Paulo Monteiro Carvalho Mori CUNHA, Lubbeek (BE); Conny Gysemans, Rotselaar (BE); Chantal Mathieu, Buizingen (BE)

(73) Assignee: Katholieke Universiteit Leuven, Leuven (BE)

(21) Appl. No.: 15/147,563

(22) Filed: May 5, 2016

Related U.S. Application Data
(60) Provisional application No. 62/157,341, filed on May 5, 2015.

(51) Int. Cl. C12N 5/071; A61K 35/39; A61K 9/16; A61K 35/28

(52) U.S. Cl. CPC C12N 5/0676; A61K 35/28; A61K 35/39; A61K 9/16; C12N 2502/03

(57) ABSTRACT

The present invention provides methods that increases the graft survival rate of pancreatic islets after pancreatic islet transplantation, maintains the survival of pancreatic islets ex vivo, and reduce the number of transplanted pancreatic islets required for normalizing blood glucose levels. When performing pancreatic islet transplantation, by contacting pancreatic islets with stem cells or by transplanting pancreatic islets and stem cells in contact with each other, it is possible to significantly improve graft survival rate of transplanted pancreatic islets and reduce the number of transplanted pancreatic islets required for normalizing blood glucose levels. The invention also provides compositions for pancreatic islet transplantation comprising the islets and the stem cells or conditioned medium from stem cell culture islets. Thus, the composition and methods are useful for treating diabetes.

### FIELD OF THE INVENTION

The present invention provides methods that increase the graft survival rate of pancreatic islets after pancreatic islet transplantation, maintains the survival of pancreatic islets ex vivo, and reduce the number of transplanted pancreatic islets required for normalizing blood glucose levels. When performing pancreatic islet transplantation, by contacting pancreatic islets with stem cells or by transplanting pancreatic islets and stem cells in contact with each other, it is possible to significantly improve graft survival rate of transplanted pancreatic islets and reduce the number of transplanted pancreatic islets required for normalizing blood glucose levels. The invention also provides compositions for pancreatic islet transplantation comprising the islets and the stem cells or conditioned medium from stem cell culture islets. Thus, the compositions and methods are useful for treating insulin deficiency, such as in diabetes.

### BACKGROUND OF THE INVENTION

To date insulin therapy is considered the gold standard for the treatment of type 1 diabetes (T1D). Nevertheless limitations persist, such as, frequent episodes of hypoglycemia and chronic micro- and macrovascular complications [Downs C A, et al. (2015) World J Diabetes 6: 554-565; Campbell M D, et al. (2015) BMJ Open Diabetes Res Care 3: e000085]. Islet transplantation offers an alternative treatment for T1D patients. Drawbacks include a limited supply of cadavers, the need for several donors for a single transplantation, and graft failure [Daoud J, et al. (2010) Cell Transplant 19: 1523-1535].

In an attempt to increase the rate of islet survival following transplantation, islets have been admixed with bone marrow-derived mesenchymal stem cells (MSC) (Rackham et al., Diabetologia, (2011) 54:1127-1135), (Borg et al., Diabetologia (2014) 57:522-531), (Solari et al., Journal of Autoimmunity, 32 (2009), 116-124). However, long population doubling time, early senescence, DNA damage during in vitro expansion, and poor engraftment after transplantation are disadvantages of MSC therapy [Wei X, et al., (2013) Acta Pharmacol Sin 34: 747-754]. Furthermore, with long-term culture expansion, MSC can become karyotypical abnormal, which may pose a risk of tumor formation. Adipose-derived stem cells have also been applied (U.S. Pat. No. 9,089,550).

### SUMMARY OF THE INVENTION

The inventors assessed the therapeutic efficacy of co-transplantation of undifferentiated human non-endothelial bone marrow-derived multipotent adult progenitor cells (MAPC) with mammalian islets as separate or composite pellets in a syngeneic marginal mass islet transplantation model.

The inventors considered the possibility that co-transplantation of MAPC might improve islet engraftment and survival. Islets were co-transplanted with or without MAPC as separate or composite pellets. In the Examples this was done by transplantation of human MAPC under the kidney capsule of syngeneic alloxan-induced diabetic C57BL/6 mice. Islet-human MAPC co-transplantation as a composite pellet significantly improved the outcome of islet transplantation as measured by the initial glycemic control, diabetes reversal rate, glucose tolerance, and serum C-peptide concentration, compared with transplantation of islets alone. Histologically, a higher blood vessel area and density, in addition to a higher vessel/islet ratio, were detected in recipients of islet-human MAPC composites.

The conclusion was that co-transplantation of pancreatic islets with MAPC could enhance islet graft revascularization and improve islet graft function.

Accordingly, the present invention provides compositions and methods to enhance the graft survival rate of islets after transplantation, to culture islets isolated from a subject for an extended period of time, and to reduce the number of transplanted islets required for normalizing blood glucose levels.

In all compositions and methods herein, the stem cells and/or pancreatic islets can be derived from humans. The invention extends to other mammals as well, e.g., dogs, cats, horses, pigs, and other domestic animals.

The present invention includes a composition for pancreatic islet transplantation comprising stem cells and pancreatic islets.

The present invention includes a kit for pancreatic islet transplantation, comprising a first cell preparation containing stem cells and a second cell preparation containing pancreatic islets.

The present invention includes a composite pellet comprising stem cells and pancreatic islets.

The present invention includes a composition comprising stem cells and pancreatic islets in cell culture medium.

The present invention includes a pharmaceutical composition comprising pancreatic islets and stem cells.

The present invention includes a composite in which stem cells are adhered to a pancreatic islet.

The present invention includes a composite in which the pancreatic islet is covered with the stem cells.

The present invention includes a method for improving pancreatic islets for transplantation, the method comprising contacting pancreatic islets with stem cells. The method can be ex vivo culturing pancreatic islets in the presence of stem cells. The method also may involve contacting the islets and stem cells in vivo (as in co-implantation). In ex vivo methods, the cells may merely be mixed (i.e., do not have to be cultured together). The mixed cells may be pelleted to a form that is convenient for transplantation (e.g., by centrifuge).

The present invention includes a method to improve islet viability ex vivo, the method comprising contacting pancreatic islets with stem cells.

The pancreatic islets can be a cultured-expanded pancreatic islet cell preparation.

The pancreatic islets and stem cells can be contacted in culture medium and cultured together for a desired period of time.

The pancreatic islets and stem cells can be contacted pre-transplantation.

The pancreatic islets and stem cells can be contacted in vivo (co-transplant).

The present invention includes a method to improve islet graft survival in vivo by contacting islets with stem cells ex vivo prior to islet administration to a subject.

The present invention includes a method to improve islet graft survival in vivo by co-administering stem cells and islets to a subject.

In a specific exemplified embodiment the ratio of islets to stem cells is about 500:250,000.

The present invention includes a pancreatic islet transplantation method, comprising simultaneous administration of pancreatic islets and stem cells to a patient in need of a treatment of diabetes, with or without pre-transplant contact of the islets with the stem cells.

The present invention includes a therapeutic method for treating diabetes, the method comprising administering the composite to a patient in need of a treatment.

The present invention includes a method for producing the composite, the method comprising co-culturing the stem cells and the pancreatic islets. The composite can also be formed by physical methods that provide contact, such as, centrifugation of the islets and stem cells, encapsulation, and the like.

The present invention includes a method for maintaining survival of pancreatic islets, the method comprising co-culturing stem cells and pancreatic islets.

The present invention includes a therapeutic method for diabetes, the method comprising the steps of (A) and (B):
(A) forming a composite of the pancreatic islet and the stem cells, such as by co-culturing a pancreatic islet and stem cells or otherwise physically contacting the islets and stem cells; and
(B) administering the composite to a patient in need of a treatment of diabetes.

The present invention includes a non-pharmaceutical composition comprising pancreatic islets and stem cells.

The present invention includes the stem cell-islet composition in a pharmaceutically-acceptable composition.

The present invention includes a composition comprising pancreatic islets and stem cells admixed with a pharmaceutically-acceptable carrier.

The present invention includes a method for making a composition by admixing stem cells and islets.

The present invention includes compositions in which the pancreatic islets have been pre-incubated with stem cells in vitro.

In all the compositions and methods, the compositions may consist essentially of the stem cells and the islets. These two components provide the therapeutic effects.

In all the compositions and methods, islets can be substituted by pancreatic β-cells, a cell type that is contained in normal islets and that secretes insulin. The cells comprise 65-80% of the cells in the islets. They store and release insulin.

Because the stem cells may provide the beneficial effects on pancreatic islets by secreted factors, the islets can also be pre-incubated with or administered with medium that has been conditioned by culturing the stem cells. This medium, accordingly, will be cell-free.

With the present invention, by mixing and co-transplanting stem cells and islets, or by using a composite of stem cells and islets for islet transplantation, graft survival rate of islets can be improved. As a result, it is possible to reduce the amount of islets required for transplantation, the number of transplantations, and effectively treat diabetes through islet transplantation.

The present invention allows the number of islets required for transplantation to be reduced; therefore, islets obtained from a single donor can be transplanted to multiple recipients. This ameliorates the shortage of donors for islets and provides an innovative technique for spreading islet transplantation into general medical care.

The present invention allows the survival of the islets ex vivo to be maintained (i.e., increase viability of the islets) by co-culturing stem cells with islets. These islets may be isolated from a living body. This makes it possible to store the islets in a state ready for islet transplantation for an extended period of time; that way, the islets isolated from a single donor can be transplanted to a more suitable recipient.

Furthermore, because the islets can be stably cultured in vitro while retaining their ability to secrete insulin, it is possible to administer an immunosuppressant to a recipient prior to conducting islet transplantation. However, since the cells of the present invention may be immunomodulatory and may not be immunogenic, in one embodiment, the islets can be administered without an immunosuppressive agent other than the stem cells themselves.

Cells include, but are not limited to, cells that are not embryonic stem cells and not germ cells, having some characteristics of embryonic stem cells, but being derived from non-embryonic tissue, and providing the effects described in this application. The cells may naturally achieve these effects (i.e., not genetically or pharmaceutically modified). However, natural expressors can be genetically or pharmaceutically modified to increase potency. In one embodiment, the stem cells can be non-HLA matched, allogeneic cells.

The cells may express pluripotency markers, such as oct4. They may also express markers associated with extended replicative capacity, such as telomerase. Other characteristics of pluripotency can include the ability to differentiate into cell types of more than one germ layer, such as two or three of ectodermal, endodermal, and mesodermal embryonic germ layers. The cells may be highly expanded without being transformed or tumorigenic and also maintain a normal karyotype. In one embodiment, the non-embryonic stem, non-germ cells may have undergone a desired number of cell doublings in culture. For example, non-embryonic stem, non-germ cells may have undergone at least 10-40 cell doublings in culture, such as 30-35 cell doublings, wherein the cells are not transformed and have a normal karyotype. The cells may differentiate into at least one cell type of each of two of the endodermal, ectodermal, and mesodermal embryonic lineages and may include differentiation into all three. Further, the cells may not be tumorigenic, such as, not producing teratomas. If cells are transformed or tumorigenic, and it is desirable to use them for infusion, such cells may be disabled so they cannot form tumors in vivo, as by treatment that prevents cell proliferation into tumors. Such treatments are well known in the art.

Cells include, but are not limited to, the following numbered embodiments:

1. Isolated expanded non-embryonic stem, non-germ cells, the cells having undergone at least 10-40 cell doublings in culture, wherein the cells express oct4, are not transformed, and have a normal karyotype.

2. The non-embryonic stem, non-germ cells of 1 above that further express one or more of telomerase, rex-1, or sox-2.

3. The non-embryonic stem, non-germ cells of 1 above that can differentiate into at least one cell type of at least two of the endodermal, ectodermal, and mesodermal embryonic lineages.

4. The non-embryonic stem, non-germ cells of 3 above that further express one or more of telomerase, rox-1, or sox-2.

5. The non-embryonic stem, non-germ cells of 3 above that can differentiate into at least one cell type of each of the endodermal, ectodermal, and mesodermal embryonic lineages.

6. The non-embryonic stem, non-germ cells of 5 above that further express one or more of telomerase, rex-1, or sox-2.

7. Isolated expanded non-embryonic stem, non-germ cells that are obtained by culture of non-embryonic, non-germ tissue, the cells having undergone at least 40 cell doublings in culture, wherein the cells are not transformed and have a normal karyotype.

8. The non-embryonic stem, non-germ cells of 7 above that express one or more of oct4, telomerase, rex-1, or sox-2.

9. The non-embryonic stem, non-germ cells of 7 above that can differentiate into at least one cell type of at least two of the endodermal, ectodermal, and mesodermal embryonic lineages.

10. The non-embryonic stem, non-germ cells of 9 above that express one or more of oct4, telomerase, rex-1, or sox-2.

11. The non-embryonic stem, non-germ cells of 9 above that can differentiate into at least one cell type of each of the endodermal, ectodermal, and mesodermal embryonic lineages.

12. The non-embryonic stem, non-germ cells of 11 above that express one or more of oct4, telomerase, rex-1, or sox-2.

13. Isolated expanded non-embryonic stem, non-germ cells, the cells having undergone at least 10-40 cell doublings in culture, wherein the cells express telomerase, are not transformed, and have a normal karyotype.

14. The non-embryonic stem, non-germ cells of 13 above that further express one or more of oct4, rex-1, or sox-2.

15. The non-embryonic stem, non-germ cells of 13 above that can differentiate into at least one cell type of at least two of the endodermal, ectodermal, and mesodermal embryonic lineages.

16. The non-embryonic stem, non-germ cells of 15 above that further express one or more of oct4, rex-1, or sox-2.

17. The non-embryonic stem, non-germ cells of 15 above that can differentiate into at least one cell type of each of the endodermal, ectodermal, and mesodermal embryonic lineages.

18. The non-embryonic stem, non-germ cells of 17 above that further express one or more of oct4, rex-1, or sox-2.

19. Isolated expanded non-embryonic stem, non-germ cells that can differentiate into at least one cell type of at least two of the endodermal, ectodermal, and mesodermal embryonic lineages, said cells having undergone at least 10-40 cell doublings in culture.

20. The non-embryonic stem, non-germ cells of 19 above that express one or more of oct4, telomerase, rex-1, or sox-2.

21. The non-embryonic stem, non-germ cells of 19 above that can differentiate into at least one cell type of each of the endodermal, ectodermal, and mesodermal embryonic lineages.

22. The non-embryonic stem, non-germ cells of 21 above that express one or more of oct4, telomerase, rex-1, or sox-2.

Additional functions of the cells that are shown in the specific examples in this application include angiogenic potential and the ability to secrete certain angiogenic proteins, including, but not limited to, one or more of VEGF, A, C, and D, PIGF, sFlt-1, bFGF, and IL8.

The cells lack expression of HLA-DR, CD45, glyA, CD34.

Since the stem cells may provide the effects described herein by means of secreted molecules, the various embodiments described herein for administration of stem cells may be done by administration of one or more of the secreted molecules, such as might be in conditioned culture medium. In one embodiment, a conditioned medium is used instead of the stem cells.

The stem cells may be prepared by the isolation and culture conditions described herein. In a specific embodiment, they are prepared by culture conditions that are described herein involving lower oxygen concentrations combined with higher serum.

It should be understood that this invention is not limited to the particular methodology, protocols, and reagents, etc., described herein and, as such, may vary. The terminology used herein is for the purpose of describing particular embodiments only, and is not intended to limit the scope of the disclosed invention, which is defined solely by the claims.

The section headings are used herein for organizational purposes only and are not to be construed as in any way limiting the subject matter described.

The methods and techniques of the present application are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990).

### BRIEF DESCRIPTION OF THE DRAWINGS

FIGS. 1A-1C: Characterization of human MAPC. (FIG. 1A) Cell surface marker expression of human bone marrow-derived MAPC. Flow cytometry histograms show the expression levels (shaded dark gray peaks) of selected markers associated with the characterization of human MAPC [CD44, CD49c, CD105] compared with negative isotype controls (shaded light gray peaks). (FIG. 1B) Culture medium of human MAPC was analyzed with human biomarker 40-Plex kit containing a pro-inflammatory panel, cytokine panel, chemokine panel, angiogenesis panel and vascular inflammation panel. (FIG. 1C) Pro-angiogenic properties of human MAPC in a chorioallantoic membrane (CAM) assay, with BSA as negative control and VEGF-A as positive control (mean±SEM, n=3-9 per group). **, P<0.01.

FIGS. 2A-2D: In vivo function of a marginal islet mass co-transplanted with human MAPC. (FIG. 2A) Blood glucose measurements of alloxan-induced diabetic C57BL/6 mice transplanted with 150 islets alone (control; white bars) or 150 islets co-transplanted as separate (SEP, dark gray bars) or composite pellets (MIX, light gray bars) with 250,000 human MAPC. *p<0.05, **p<0.01, ***p<0.001 versus islet-alone group (control), (FIG. 2B) Percentage of cured (back bars) and non-cured (gray bars) mice after islet transplantation, (FIG. 2C, FIG. 2D) Area under the curve (AUC) and blood glucose measurements of IPGTTs in all mice 2 (FIG. 2C) and 5 (FIG. 2D) weeks after transplantation, *p<0.05, **p<0.01 versus islet-alone group (control).

FIGS. 3A-3C: Morphology and composition of islets co-transplanted with human MAPC 2 and 5 weeks post-transplantation. (FIG. 3A) Box and whiskers plots of mRNA levels of mouse insulin, glucagon, and somatostatin in isolated islet grafts. Data are expressed as relative value compared to house-keeping genes. Statistical analysis was calculated using Mann-Whitney t-tests. *p<0.05. (FIG. 3B) Box and whiskers plots of volumes of beta-, alpha- and delta-cells of grafts derived from mice transplanted with a marginal-islet mass alone or combined with human MAPC as separate or composite pellet. Statistical analysis was calculated using Mann-Whitney t-tests. (FIG. 3C) Distribution of mouse insulin-(white), glucagon-(red), and somatostatin (green)-positive cells in islet grafts composed of islet-human MAPC as separate (SEP) or composite (MIX) pellets or of islets alone (control) at 2 weeks post-transplantation. Images are representative of sections from 2-6 different animals Scale bar is 100 μm.

FIGS. 4A-4B: Co-transplantation of islets with human MAPC as composites promotes graft revascularization in a marginal islet mass diabetic mouse model. (FIG. 4A) Representative sections of 5-week grafts consisting of mouse islets transplanted alone or with human MAPC as separate (SEP) or composite (MIX) pellet. Images are representative of insulin and endomucin (vessel) staining for 3-4 animals in each transplant group. Scale bar is 100 μm. (FIG. 4B) Vessel morphologic parameters assessment was determined as described in material and methods section. Data are means±SEM. Statistical analysis was calculated using Mann-Whitney t-tests. *p<0.05, **p<0.01.

FIGS. 5A-5B: Non-fasting glycemia and body weight of transplant recipients. (FIG. 5A) Blood glucose concentrations were monitored in alloxan-induced diabetic C57BL/6 mice transplanted with 150 syngeneic islets either alone or co-transplanted with 250,000 human MAPC for over 5 weeks. Recovery nephrectomies performed in randomly selected animals of each group at 5 weeks post-transplant resulted in 100% return to hyperglycemia. (FIG. 5B) Body weight changes did not significantly differ between the various groups throughout the study period. Each value represents the mean±SEM.

FIGS. 6A-6B: Serum insulin and C-peptide concentrations 2 (FIG. 6A) and 5 weeks (FIG. 6B) post-transplantation. Mice were transplanted with 150 islets alone (white bars) or with 150 islets together with human MAPC as separate pellets (SEP, dark gray bars) or as a composite pellet (MIX, light gray bars). **p<0.01 versus islet-alone group (control).

### DETAILED DESCRIPTION OF THE INVENTION

### Definitions

“A” or “an” means herein one or more than one; at least one. Where the plural form is used herein, it generally includes the singular.

A “cell bank” is industry nomenclature for cells that have been grown and stored for future use. Cells may be stored in aliquots. They can be used directly out of storage or may be expanded after storage. This is a convenience so that there are “off the shelf” cells available for administration. The cells may already be stored in a pharmaceutically-acceptable excipient so they may be directly administered or they may be mixed with an appropriate excipient when they are released from storage. Cells may be frozen or otherwise stored in a form to preserve viability. In one embodiment of the invention, cell banks are created in which the cells have been selected for enhanced potency to achieve the effects described in this application. Following release from storage, and prior to administration, it may be preferable to again assay the cells for potency. This can be done using any of the assays, direct or indirect, described in this application or otherwise known in the art. Then cells having the desired potency can then be administered. Banks can be made using autologous cells (derived from the organ donor or recipient). Or banks can contain cells for allogeneic uses.

“Co-administer” with respect to this invention means to administer together two or more agents. Within the context of the present invention, in one embodiment the stem cells and the pancreatic islets are administered in the same pharmaceutical composition so that the pancreatic islets and the stem cells contact each other in this composition. However, it is possible, particularly in a case of local administration, that the islets or the stem cells might be administered first and then the islets or stem cells would be administered later but in such a way that the stem cells can still contact the islets in order to produce the beneficial effect on the islets. It is also understood that the stem cells can be replaced with conditioned media produced by culturing the cells that contain the factors that have the beneficial effect on the islets.

A “composite pellet” is a composition that comprises both stem cells and pancreatic islets in direct physical contact. Pharmaceutical compositions comprising these composites consist essentially of stem cells and the pancreatic islets in a pharmaceutically-acceptable carrier. The islets themselves may be covered with the stem cells. The stem cells may be directly adhered to the islets. In some embodiments substantially the entire surface of the islet is covered with the stem cells.

The method for producing the composite comprises any method by which the cells are mixed together so that they can be administered and will remain in contact with each other. The method may consist essentially of co-culturing or mixing the stem cells with the pancreatic islets.

Composites are formed containing both the islets and the stem cells in a form so that they can be administered in physical contact with each other. In one embodiment the islets and the stem cells are cultured prior to administration. For example, they both may be seeded on to a plate and cultured for a desirable amount of time. The time may be of short duration, for example, 5 minutes. Or it may be longer, for example, up to 24 hours or longer. Where the goal is to provide a composite in which the stem cells coat and adhere to the islets, the culture can be observed so that the degree of coating/adherence that is desired can be ascertained. Thus, the effective time can be variable.

Although the ratio and absolute amount of cells that are contained in the composite may vary depending on the specific circumstances of the subject, in some embodiments where the cells are seeded into a multi-well plate, each well can contain about 50 islets and about 10,000 stem cells. The amount of islets and stem cells in the islets are configured, preferably, to provide normal glucose levels, which are approximately a concentration of 100 mg/dL. The composite in the Example in this application comprised about 150 islets and 250,000 stem cells.

In some embodiments, the islets and stem cells are used to form the composite immediately before administration, that is, with no significant prior contact in culture or otherwise. So contact can be, for example, for even less than 5 minutes (such as in the Example). The composites that are thus formed can then be used in co-transplantation of the islets and the cells in order to treat islet deficiency, as in diabetes (i.e., improve blood glucose levels).

Composites can be formed by methods that are known in the art, such as, Johansson et al., Diabetes 57:2393-2401 (2008), Ito et al., Transplantation 89:1438-1445 (2010), Sakata et al., Transplantation 89:686-693 (2020), Ohmura et al., Transplantation 90:1366-1373 (2010), Solari et al., J Autoimmunity 32:116-124 (2009), Rackham et al., Diabetologia 54:1127-1135 (2011), Borg et al., Diabetologia 57:522-531 (2014), Hajizedeh-Saffar et al., Sci Rep 5:9322 (2015). All of the above are incorporated by reference for teaching the production of composites. As is indicated by these references the formation of composites of islets and other cell types can be accomplished by methods that are known in the art.

The number of stem cells is that which provides improved glucose levels per number of islets when compared to the administration of the same number of islets alone. So, for example, if 100 islets produce a certain blood glucose level and the effect of stem cells is to produce that same level with less than that number of islets, or to produce better glucose levels with that same number of islets, that would constitute an “improvement”. The end goal is to decrease the number of islets that is necessary to achieve blood glucose levels within the normal range.

An islet comprises about 1,000-2,000 cells (including α, β, γ cells). The isolation of the pancreatic islets can be made by methods that are well known in the art, such as, Johansson et al., Am J Transplant 5:2632-2639 (2005), incorporated by reference for this procedure.

In all methods and compositions, instead of whole islets, cultured pancreatic β cells can be used to provide the same effects.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning.

“Comprised of” is a synonym of “comprising” (see above).

The term “contact”, when used in relation to a stem cell and the islets to be transplanted, can mean that, upon exposure to the islets, the stem cell physically touches the islets. In such instances, the stem cell is in direct physical contact with the islets. In other instances, the stem cell can indirectly contact the islets, where one or more structures (e.g., another cell) and/or fluids (e.g., blood) physically intervene(s) between the stem cell and the islets.

“Effective amount” generally means an amount which achieves the specific desired effects described in this application. For example, an effective amount is an amount sufficient to effectuate a beneficial or desired clinical result. Within the context of this invention generally the desired effect is a clinical improvement compensating for the ineffective or pathological function of the islets present in a subject. In one embodiment the subject has diabetes and the effect is to improve or completely normalize blood glucose levels. The effective amounts can be provided all at once in a single administration or in fractional amounts that provide the effective amount in several administrations. The precise determination of what would be considered an effective amount may be based on factors individual to each subject, including the severity of the disease/deficiency, health of the patient, age, etc. One skilled in the art will be able to determine the effective amount based on these considerations which are routine in the art. As used herein, “effective dose” means the same as “effective amount.”

Accordingly, an effective amount of the islets is that in which the clinical symptoms of the subject are improved. And an effective amount of stem cells would be that which is sufficient to produce islets that provide that improved clinical outcome.

“Effective route” generally means a route which provides for delivery of an agent to a desired compartment, system, or location. For example, an effective route is one through which an agent can be administered to provide at the desired site of action an amount of the agent sufficient to effectuate a beneficial or desired clinical result (in the present case, effective transplantation).

The term “exogenous”, when used in relation to a stem cell, generally refers to a stem cell that is external to the subject and which has been exposed to (e.g., contacted with) the islets that are intended for transplantation by an effective route. An exogenous stem cell may be from the same subject or from a different subject. In one embodiment, exogenous stem cells can include stem cells that have been harvested from a subject, isolated, expanded ex vivo, and then exposed to the islets intended for transplantation by an effective route.

The term “expose” can include the act of contacting one or more stem cells with the islets intended for transplantation. Contacting the islets can be done ex vivo or in vivo (e.g., by providing stem cells to the islets in the subject, such as, in local administration).

Use of the term “includes” is not intended to be limiting.

“Increase” or “increasing” means to induce a biological event entirely or to increase the degree of the event.

The term “isolated” refers to a cell or cells which are not associated with one or more cells or one or more cellular components that are associated with the cell or cells in vivo. An “enriched population” means a relative increase in numbers of a desired cell relative to one or more other cell types in vivo or in primary culture.

However, as used herein, the term “isolated” does not indicate the presence of only the cells of the invention. Rather, the term “isolated” indicates that the cells of the invention are removed from their natural tissue environment and are present at a higher concentration as compared to the normal tissue environment. Accordingly, an “isolated” cell population may further include cell types in addition to the cells of the invention cells and may include additional tissue components. This also can be expressed in terms of cell doublings, for example. A cell may have undergone 10, 20, 30, 40 or more doublings in vitro or ex vivo so that it is enriched compared to its original numbers in vivo or in its original tissue environment (e.g., bone marrow, peripheral blood, placenta, umbilical cord, umbilical cord blood, etc.).

“MAPC” is an acronym for “multipotent adult progenitor cell.” It refers to a cell that is not an embryonic stem cell or germ cell but has some characteristics of these. MAPC can be characterized in a number of alternative descriptions, each of which conferred novelty to the cells when they were discovered. They can, therefore, be characterized by one or more of those descriptions. First, they can have extended replicative capacity in culture without being transformed (tumorigenic) and with a normal karyotype. Second, they may give rise to cell progeny of more than one germ layer, such as two or all three germ layers (i.e., endoderm, mesoderm and ectoderm) upon differentiation. Third, although they are not embryonic stem cells or germ cells, they may express markers of these primitive cell types so that MAPCs may express one or more of Oct 3/4 (i.e., Oct4, Oct 3A). Fourth, like a stem cell, they may self-renew, that is, have an extended replication capacity without being transformed. This means that these cells express telomerase (i.e., have telomerase activity). Accordingly, the cell type that was designated “MAPC” may be characterized by alternative basic characteristics that describe the cell via some of its novel properties.

The term “adult” in MAPC is non-restrictive. It refers to a non-embryonic somatic cell as above. MAPCs are karyotypically normal and do not form teratomas in vivo. This acronym was first used in U.S. Pat. No. 7,015,037 to describe a cell isolated from bone marrow that had extensive replicative capacity and expressed pluripotency markers.

MAPC represents a more primitive progenitor cell population than MSC (Verfaillie, C. M., Trends Cell Biol 12:502-8 (2002), Jahagirdar, B. N., et al., Exp Hematol, 29:543-56 (2001); Reyes, M. and C. M. Verfaillie, Ann NY Acad Sci, 938:231-233 (2001); Jiang, Y. et al., Exp Hematol, 30896-904 (2002); and Jiang, Y. et al., Nature, 418:41-9. (2002)).

The term “MultiStem®” is the trade name for a cell preparation based on the MAPCs of U.S. Pat. No. 7,015,037, i.e., a non-embryonic stem, non-germ cell as described above. MultiStem® is prepared according to cell culture methods disclosed in this patent application, particularly, lower oxygen and higher serum. MultiStem® is highly expandable, karyotypically normal, and does not form teratomas in vivo. It may differentiate into cell lineages of more than one germ layer and may express telomerase.

“Pharmaceutically-acceptable carrier” is any pharmaceutically-acceptable medium for the cells and/or islets used in the present invention. Such a medium may retain isotonicity, cell metabolism, pH, and the like. It is compatible with administration to a subject and can be used, therefore, for islet and/or cell delivery and treatment.

“Progenitor cells” are cells produced during differentiation of a stem cell that have some, but not all, of the characteristics of their terminally-differentiated progeny. Defined progenitor cells, such as “cardiac progenitor cells,” are committed to a lineage, but not to a specific or terminally differentiated cell type. The term “progenitor” as used in the acronym “MAPC” does not limit these cells to a particular lineage. A progenitor cell can form a progeny cell that is more highly differentiated than the progenitor cell.

The term “reduce” as used herein means to prevent as well as decrease. In the context of treatment, to “reduce” is to either prevent or ameliorate the deficiency. This includes causes or symptoms of islet deficiency.

“Selecting” a cell with a desired level of potency can mean identifying (as by assay), isolating, and expanding a cell. This could create a population that has a higher potency than the parent cell population from which the cell was isolated. The “parent” cell population refers to the parent cells from which the selected cells divided. “Parent” refers to an actual P1→F1 relationship (i.e., a progeny cell). So if cell X is isolated from a mixed population of cells X and Y, in which X is an expressor and Y is not, one would not classify a mere isolate of X as having enhanced expression. But, if a progeny cell of X is a higher expressor, one would classify the progeny cell as having enhanced expression.

To select a cell that achieves the desired effect would include both an assay to determine if the cells achieve the desired effect and would also include obtaining those cells. The cell may naturally achieve the desired effect in that the effect is not achieved by an exogenous transgene/DNA. But an effective cell may be improved by being incubated with or exposed to an agent that increases the effect. The cell population from which the effective cell is selected may not be known to have the potency prior to conducting the assay. The cell may not be known to achieve the desired effect prior to conducting the assay. As an effect could depend on gene expression and/or secretion, one could also select on the basis of one or more of the genes that cause the effect.

Selection could be from cells in a tissue. For example, in this case, cells would be isolated from a desired tissue, expanded in culture, selected for achieving the desired effect, and the selected cells further expanded.

Selection could also be from cells ex vivo, such as cells in culture. In this case, one or more of the cells in culture would be assayed for achieving the desired effect and the cells obtained that achieve the desired effect could be further expanded.

Cells could also be selected for enhanced ability to achieve the desired effect. In this case, the cell population from which the enhanced cell is obtained already has the desired effect. Enhanced effect means a higher average amount per cell than in the parent population.

The parent population from which the enhanced cell is selected may be substantially homogeneous (the same cell type). One way to obtain such an enhanced cell from this population is to create single cells or cell pools and assay those cells or cell pools to obtain clones that naturally have the enhanced (greater) effect (as opposed to treating the cells with a modulator that induces or increases the effect) and then expanding those cells that are naturally enhanced.

However, cells may be treated with one or more agents that will induce or increase the effect. Thus, substantially homogeneous populations may be treated to enhance the effect.

If the population is not substantially homogeneous, then, it is preferable that the parental cell population to be treated contains at least 100 of the desired cell type in which enhanced effect is sought, more preferably at least 1,000 of the cells, and still more preferably, at least 10,000 of the cells. Following treatment, this sub-population can be recovered from the heterogeneous population by known cell selection techniques and further expanded if desired.

Thus, desired levels of effect may be those that are higher than the levels in a given preceding population. For example, cells that are put into primary culture from a tissue and expanded and isolated by culture conditions that are not specifically designed to produce the effect may provide a parent population. Such a parent population can be treated to enhance the average effect per cell or screened for a cell or cells within the population that express greater degrees of effect without deliberate treatment. Such cells can be expanded then to provide a population with a higher (desired) expression.

“Self-renewal” of a stem cell refers to the ability to produce replicate daughter stem cells having differentiation potential that is identical to those from which they arose. A similar term used in this context is “proliferation.”

“Stem cell” means a cell that can undergo self-renewal (i.e., progeny with the same differentiation potential) and also produce progeny cells that are more restricted in differentiation potential. Within the context of the invention, a stem cell would also encompass a more differentiated cell that has de-differentiated, for example, by nuclear transfer, by fusion with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

Dedifferentiation may also be caused by the administration of certain compounds or exposure to a physical environment in vitro or in vivo that would cause the dedifferentiation. Stem cells also may be derived from abnormal tissue, such as a teratocarcinoma and some other sources such as embryoid bodies (although these can be considered embryonic stem cells in that they are derived from embryonic tissue, although not directly from the inner cell mass). Stem cells may also be produced by introducing genes associated with stem cell function into a non-stem cell, such as an induced pluripotent stem cell.

“Subject” means a vertebrate, such as a mammal, such as a human Mammals include, but are not limited to, humans, dogs, cats, horses, cows, and pigs.

The term “therapeutically effective amount” refers to the amount of an agent determined to produce any therapeutic response in a mammal. For example, effective therapeutic agents may prolong the survivability of the patient, and/or inhibit overt clinical symptoms. Treatments that are therapeutically effective within the meaning of the term as used herein, include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any pathological symptoms. In one aspect, treatment means to improve blood glucose levels, i.e., towards or in normal ranges.

In the context of the invention a therapeutically effective amount is that amount of stem cells that beneficially affect the islet cells to the extent that transplantation of the islet cells results in an improvement in the clinical outcome (e.g., blood glucose levels). Also, an effective amount of stem cells is that which improves the survivability of islet cells ex vivo prior to transplantation and/or the survivability of the islets in the subject after transplantation. An effective amount of stem cells can also be that amount that is co-administered with the islets to a subject to achieve a therapeutic outcome. Accordingly, a therapeutically effective amount of the islets is also that number of islets that can achieve that improved clinical outcome upon transplantation. The effective amounts of stem cells and islets can be determined by routine empirical experimentation.

The term “therapeutically effective time” can refer to the time necessary to contact the islets with the stem cells in order to achieve the clinical improvement (i.e., improve blood glucose levels). For example, if the cells and islets are contacted in vitro, (ex vivo), an effective time is that which provides for improved survivability of the islets which results in a positive therapeutic outcome. This time could be in the range of 5-10 minutes up to several hours or even longer. Examples are 15-30 minutes, 30-45 minutes, and 45 minutes to an hour. When stem cells are cultured together with islets, the time can be longer, e.g., 24 hours or more. It depends on how long it takes the stem cells to coat/adhere to the islets.

A therapeutically effective time could also refer to the time required for a subject to receive the islets and achieve an improved clinical status (e.g., improved blood glucose levels).

The term “therapeutically effective route” refers to the routes of administration that may be effective for achieving an improved clinical outcome. The therapeutically effective route means that the stem cells mixed with the islets would be co-transplanted at whatever site the islets can produce their beneficial effect. Local administration, such as, under the kidney capsule is an example. However, islet transplantation (along with the stem cells) can be done by any of the effective routes that are known in the art. In humans this can be via intraportal implantation.

In determining an appropriate amount of stem cells to achieve the beneficial effects on a given amount of islets is determined empirically on the basis of providing the islets with the ability to achieve improved glycemic indexes after transplantation, such as, even normal glycemic indexes. As exemplified herein, a dose range for the composite could be 250,000-500,000 stem cells. Thus, these amounts need to be determined empirically based on the method of delivery, the severity of the illness, and the like.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

“Validate” means to confirm. In the context of the invention, one confirms that a cell has a desired potency for beneficially affecting the islets in vivo or in vitro. This is so that one can then use that cell (in treatment, banking, drug screening, etc.) with a reasonable expectation of efficacy. Accordingly, to validate means to confirm that the cells, having been originally found to have/established as having the desired activity, in fact, retain that activity. Thus, validation is a verification event in a two-event process involving the original determination and the follow-up determination. The second event is referred to herein as “validation.”

Pancreatic islets, which are also referred to as Langerhans islets, are cells (or lumps of cells) ordinarily having a size of 100 to 200 μm. Their main constituent cells include α-cells that secrete glucagon, β-cells that secrete insulin, δ-cells that secrete somatostatin, and PP-cells that secrete pancreatic polypeptides. The origin (donor) of the islets that are to be transplanted may be a mammal of the same species as a recipient, and, for example, when the recipient is human, human-derived islets are used.

In addition, specific examples of the method for isolating islets from a pancreas include a method that may have the following steps of (i) to (iii). Furthermore, when the donor is human, a method based on the Ricordi method, known in the art, can be illustrated as an example.

(i) An enzyme such as collagenase is allowed to uniformly penetrate and swell a pancreas. The present step is preferably performed in a low temperature condition of about 4 to 6° C. in order to prevent enzyme reaction to proceed.

(ii) The swelled pancreas is digested through enzyme reaction. The digestion through enzyme reaction is performed by exposing the swelled pancreas to a temperature that allows enzyme reaction to proceed (e.g., about 37° C.). In addition to digesting the swelled pancreas using an enzyme, the pancreas may be mechanically decomposed through vibration or the like if necessary.

(iii) Using density gradient centrifugation, islets are isolated from the cell population obtained through enzyme digestion. In the density gradient centrifugation, a part from which islets can be obtained is suitably selected depending on the rotational velocity of the centrifugal separation, the type of density gradient solution, etc.

The isolated islets may be stored in an appropriate preservation solution if necessary. The isolated islets are preferably cultured and stored together with the stem cells. By doing so, the isolated islets can be maintained in a living state for a further extended period of time.

The compositions may include, other than the stem cells, a carrier that is pharmaceutically acceptable and that does not adversely affect the stem cells. Examples of such carrier include saline, PBS, culture media, protein pharmaceuticals, including albumin, solutions for pancreatic islet preservation, etc.

The administration dose of the stem cells is selected as appropriate in accordance with the transplantation route, presence of a composite formed with an islet, the quantity of islets that are to be transplanted, the severity of the symptom of a recipient, etc. For example, when the stem cells, in a state of not forming a composite with an islet are mixed together with islets and injected to a recipient having a body weight of 50 kg at a location under the renal capsule, in greater omentum, or in subcutaneous tissue; the administration dose for a single islet transplantation is, for example, 5.0×10⁷ to 1.0×10⁹ cells, and is preferably 1.0×10⁸ to 5.0×10⁸ cells. When the stem cells are administered intraportally in the form of a composite to a recipient having a body weight of 50 kg; the administration dose for a single islet transplantation is, for example, 1.0×10⁸ to 2.0×10⁹ cells, and is preferably 5.0×10⁸ to 1.0×10⁹ cells. Therefore, the invention can include the number of stem cells that allows the quantity of islets for a single transplantation to be within the above-described range.

The administration dose for the stem cells the ratio of to-be-transplanted islets are also suitably selected in accordance with the transplantation route, the quantity of islets to be transplanted, the severity of the symptom of a recipient, etc. For example, when the stem cells are mixed with islets and injected at a location under the renal capsule, in greater omentum, or in subcutaneous tissue; with regard to the ratio of the number of transplanted islets with respect to the number of stem cells, stem cells:islets is, for example, 400:1 to 3000:1 or 500:1 to 2000:1, or is preferably 600:1 to 1500:1. Furthermore, when the stem cells are injected into a portal vein; with regard to the ratio of the number of transplanted islets with respect to the number of stem cells, stem cells:islets is, for example, 500:1 to 3500:1 or 1000:1 to 3000:1, or is preferably 1500:1 to 2500:1. Based on this, the ratio based on cell number can be obtained, since an islet normally consists of approximately 1000 to 2000 islets.

The quantity of islets transplanted together with the stem cells of the present invention is selected as appropriate in accordance with the severity of the symptom of a recipient, etc. Generally, the number of transplanted islets for a single islet transplantation for a recipient having a body weight of 50 kg is normally sufficient when the number is within a range of 5.0×10⁴ to 1.0×10⁷. However, since the graft survival rate for islets can be increased when the stem cells of the present invention are used, it is possible to obtain sufficient insulin independence even when the number of transplanted islets for a single islet transplantation is reduced to 1×10⁵ to 2×10⁶, preferably to 5×10⁵ to 1.5×10⁶, and further preferably to 1×10⁶ to 1.5×10⁶ with respect to a 50 kg adult patient. With such number of transplanted islets, it becomes possible to transplant islets obtained from a single donor to multiple recipients.

As long as the stem cells of the present invention are administered together with islets that are to be transplanted, there is no particular limitation in the administration mode; and the stem cells may be administered in a state of being mixed with islets, or the stem cells may be administered alone before or after islets are administered. From a standpoint of further improving graft survival of islets, preferably, the stem cells administered in a state of being mixed with islets, i.e., transplanting a cell preparation obtained by mixing stem cells and islets, and, more preferably, the stem cells are administered as the later described composite in which an islet and stem cells are adhered to each other.

As long as the stem cells of the present invention are capable of enhancing/improving graft survival of islets, there is no particular limitation in its administration route, and the administration route may be direct injection (transplantation) in blood in a portal vein or the like, or transplantation in nonvascular tissues such as in subcutaneous tissue, in greater omentum, under the renal capsule, or the like. When the stem cells of the present invention are administered as the later described composite of an islet and stem cells, injection in blood in a portal vein or the like is preferable; and when the stem cells are administered separately from islets, simultaneous injection of a mixture of islets and stem cells at a location under the renal capsule, in greater omentum or in subcutaneous tissue is preferable.

Although the stem cells of the present invention are administered to patients (recipients) whose insulin function of islet graft is reduced or lost, e.g., patients of type 1 diabetes or the like who require islet transplantation; the stem cells have expectation of being applied to patients of type 2 diabetes (brittle type) etc., and further, the stem cells are expected to be effective when applied to diabetics overall. Preferable patients are type 1 diabetics who require islet transplantation.

### Biopharmaceutical for Pancreatic Islet Transplantation

A biopharmaceutical for pancreatic islet transplantation includes the stem cells described in this application and islets that are to be transplanted.

The biopharmaceutical for pancreatic islet transplantation has the above-described stem cells and islets to be transplanted mixed therein, and may be a biopharmaceutical enabling co-transplantation of these cells as a mixture. Furthermore, the biopharmaceutical for pancreatic islet transplantation may be a biopharmaceutical for pancreatic islet transplantation including the later-described composite (may be referred to as “composite graft” or “composite pellet”) in which stem cells are adhered to an islet.

The biopharmaceutical for pancreatic islet transplantation may include, other than the stem cells and islets to be transplanted or the above described composite, a carrier that is pharmaceutically acceptable and that does not adversely affect these cells. Specific examples of such carrier include saline, PBS, culture media, protein pharmaceuticals, such as albumin, solutions for pancreatic islet preservation, etc.

With regard to the biopharmaceutical for pancreatic islet transplantation, the description above. also applies for the administration quantity (transplantation quantity) of the stem cells and islets, the ratio of these cells, the administration method of the biopharmaceutical, patients to whom the biopharmaceutical is administered, etc.

Composites in which Stem Cells are Adhered to a Pancreatic Islet

The present invention relates to a composite (composite graft) in which the stem cells are adhered to the islet.

As long as stem cells are directly adhered to an islet, there is no particular limitation in the structure of the composite; and, preferably, the composite has a structure in which at least one part of an islet is covered with stem cells. More preferably, when observed through microscopy, not less than 30%, preferably not less than 40%, more preferably not less than 50%, further preferably not less than 60%, more further preferably not less than 70%, even further preferably not less than 80%, and even more further preferably not less than 90% of the surface of an islet is covered with stem cells. Particularly preferably, the whole surface of an islet is covered with stem cells.

There is no particular limitation in the ratio of the numbers of stem cells and islets forming a single composite. But, ordinarily, the composite has a structure in which a single islet is covered with a large number of stem cells. There is no particular limitation in the number of stem cells adhering onto a single islet, and, for example, the number is ordinarily not less than 10, preferably not less than 20, more preferably not less than 30, further preferably not less than 40, and more further preferably not less than 50. Furthermore, there is no particular limitation in the number of stem cells adhering onto a single islet, and, assuming the number required for total coverage, the number is ordinarily, for example, not more than 5000, preferably not more than 4500, more preferably not more than 3000, and further preferably not more than 2500.

By having an islet and stem cells exist in close contact, the composite of the present invention can more effectively exert the survival effect and graft survival improving effect. More specifically, in order to improve the graft survival rate of islet transplantation using stem cells, it is preferably to have stem cells exist at the location where an islet is grafted to survive, and to have the islet be alive. Since stem cells are adhered onto an islet in the composite, stem cells will inevitably exist at the location where the islet forming the composite is grafted to survive. This promotes graft survival of the islet and promotes viability of the islet.

There is no particular limitation in the method for producing the composite in which stem cells are adhered to islets, and, for example, the composite can be obtained by co-culturing islets and stem cells. There is no particular limitation in the culturing time as long as the composite is formed, and the time is ordinarily 10 to 48 hours, preferably 18 to 48 hours, and more preferably 24 to 36 hours.

There is no particular limitation in the ratio of the numbers of stem cells and islets added to a culture medium to form the composite, and, ordinarily, pancreatic islets: stem cells is 1:10 to 5000, preferably 1:100 to 4500, more preferably 1:300 to 4000, further preferably 1:500 to 3500, and even further preferably 1:800 to 3000.

It will be understood that the above discussion of composites formed by co-culture applies to composites that are not made by co-culture (e.g., as in Applicants' Example, e.g., ratios).

### Therapeutic Method for Diabetes and Pancreatic Islet Transplantation Method

The present invention includes a method for treating diabetes by co-administering islets and stem cells and transplanting islets to a patient requiring a treatment for diabetes. There is no particular limitation in the patient as long as the patient requires a treatment for diabetes, and examples of such patient include type 1 diabetics, type 2 diabetics, and the like. Preferably, the patient is a type 1 diabetic requiring islet transplantation for treating diabetes.

Co-administration of islets and stem cells means administration of both of them in a single islet transplantation operation. Therefore, co-administration includes not only administration of a mixture of islets and stem cells, but also includes administration of either islets or stem cells in advance, and then administration of the other. Furthermore, co-administration also includes administration of the composite. Administration of the composite is preferable.

As long as graft survival of islets can be enhanced/improved, there is no particular limitation in the administration route of the islets and stem cells, and, for example, the administration route may be injection in blood in a portal vein or the like, or direct injection to nonvascular tissues such as in subcutaneous tissue, in greater omentum, under the renal capsule, or the like. When administered as the composite, injecting the cells in a portal vein or the like is preferable; and when the stem cells are administered separately from islets, injection at a location under the renal capsule, in greater omentum, or in subcutaneous tissue is preferable.

When administering islets and stem cells successively or in a mixed state, the administration dose for a single islet transplantation is as described in the above. There is no particular limitation in the administration dose when administering the composite state as long as the therapeutic effect is obtained. For example, the number of the composites administered for a single islet transplantation may be, ordinarily, within a range of 5.0×10⁴ to 1.0×10⁶ when administration is performed to a portal vein of a recipient having a body weight of 50 kg. However, since the graft survival rate for islets can be increased when the stem cells of the present invention are used, it is possible to obtain sufficient insulin independence even when the number of transplanted composites for a single islet transplantation is reduced to 1×10⁵ to 2×10⁶, preferably to 5×10⁵ to 1.5×10⁶, and further preferably to 1×10⁶ to 1.5×10⁶ with respect to a 50 kg adult patient. With such number of transplanted islets, it becomes possible to transplant islets obtained from a single donor to multiple recipients.

### Stem Cells

The present invention can be practiced, preferably, using stem cells of vertebrate species, such as humans, non-human primates, domestic animals, livestock, and other non-human mammals. These include, but are not limited to, those cells described below.

### Transcription Factors

A number of transcription factors and exogenous cytokines have been identified that influence the potency status of stem cells in vivo. The first transcription factor to be described that is involved in stem cell pluripotency is Oct4. Oct4 belongs to the POU (Pit-Oct-Unc) family of transcription factors and is a DNA binding protein that is able to activate the transcription of genes, containing an octameric sequence called “the octamer motif” within the promoter or enhancer region. Oct4 is expressed at the moment of the cleavage stage of the fertilized zygote until the egg cylinder is formed. The function of Oct3/4 is to repress differentiation inducing genes (i.e., FoxaD3, hCG) and to activate genes promoting pluripotency (FGF4, Utf1, Rex1). Sox2, a member of the high mobility group (HMG) box transcription factors, cooperates with Oct4 to activate transcription of genes expressed in the inner cell mass. It is essential that Oct3/4 expression in embryonic stem cells is maintained between certain levels. Over-expression or down-regulation of >50% of Oct4 expression level will alter embryonic stem cell fate, with the formation of primitive endoderm/mesoderm or trophectoderm, respectively. In vivo, Oct4 deficient embryos develop to the blastocyst stage, but the inner cell mass cells are not pluripotent. Instead they differentiate along the extraembryonic trophoblast lineage. Sall4, a mammalian Spalt transcription factor, is an upstream regulator of Oct4, and is therefore important to maintain appropriate levels of Oct4 during early phases of embryology. When Sall4 levels fall below a certain threshold, trophectodermal cells will expand ectopically into the inner cell mass. Another transcription factor required for pluripotency is Nanog, named after a celtic tribe “Tir Nan Og”: the land of the ever young. In vivo, Nanog is expressed from the stage of the compacted morula, is subsequently defined to the inner cell mass and is downregulated by the implantation stage. Downregulation of Nanog may be important to avoid an uncontrolled expansion of pluripotent cells and to allow multilineage differentiation during gastrulation. Nanog null embryos, isolated at day 5.5, consist of a disorganized blastocyst, mainly containing extraembryonic endoderm and no discernible epiblast.

### Isolation and Growth of MAPCs

Methods of MAPC isolation are known in the art. See, for example, U.S. Pat. No. 7,015,037, and these methods, along with the characterization (phenotype) of MAPCs, are incorporated herein by reference. MAPCs can be isolated from multiple sources, including, but not limited to, bone marrow, placenta, umbilical cord and cord blood, muscle, brain, liver, spinal cord, blood or skin. It is, therefore, possible to obtain bone marrow aspirates, brain or liver biopsies, and other organs, and isolate the cells using positive or negative selection techniques available to those of skill in the art, relying upon the genes that are expressed (or not expressed) in these cells (e.g., by functional or morphological assays such as those disclosed in the above-referenced applications, which have been incorporated herein by reference).

MAPCs have also been obtained my modified methods described in Breyer et al., Experimental Hematology, 34:1596-1601 (2006) and Subramanian et al., Cellular Programming and Reprogramming: Methods and Protocols; S. Ding (ed.), Methods in Molecular Biology, 636:55-78 (2010), incorporated by reference for these methods.

MAPCs from Human Bone Marrow as Described in U.S. Pat. No. 7,015,037

MAPCs do not express CD45 or glycophorin-A (Gly-A). The mixed population of cells was subjected to a Ficoll Hypaque separation. The cells were then subjected to negative selection using anti-CD45 and anti-Gly-A antibodies, depleting the population of CD45+ and Gly-A+ cells, and the remaining approximately 0.1% of marrow mononuclear cells were then recovered. Cells could also be plated in fibronectin-coated wells and cultured as described below for 2-4 weeks to deplete the cells of CD45+ and Gly-A+ cells. In cultures of adherent bone marrow cells, many adherent stromal cells undergo replicative senescence around cell doubling 30 and a more homogenous population of cells continues to expand and maintains long telomeres.

### Additional Culture Methods

In additional experiments, the density at which MAPCs are cultured can vary from about 100 cells/cm² or about 150 cells/cm² to about 10,000 cells/cm², including about 200 cells/cm² to about 1500 cells/cm² to about 2000 cells/cm². The density can vary between species. Additionally, optimal density can vary depending on culture conditions and source of cells. It is within the skill of the ordinary artisan to determine the optimal density for a given set of culture conditions and cells.

Also, effective atmospheric oxygen concentrations of less than about 10%, including about 1-5% and, especially, 3-5%, can be used at any time during the isolation, growth and differentiation of MAPCs in culture.

Cells may be cultured under various serum concentrations, e.g., about 2-20%. Fetal bovine serum may be used. Higher serum may be used in combination with lower oxygen tensions, for example, about 15-20%. Cells need not be selected prior to adherence to culture dishes. For example, after a Ficoll gradient, cells can be directly plated, e.g., 250,000-500,000/cm². Adherent colonies can be picked, possibly pooled, and expanded.

In one embodiment, used in the experimental procedures in the Examples, high serum (around 15-20%) and low oxygen (around 3-5%) conditions were used for the cell culture. Specifically, adherent cells from colonies were plated and passaged at densities of about 1700-2300 cells/cm² in 18% serum and 3% oxygen (with PDGF and EGF).

In an embodiment specific for MAPCs, supplements are cellular factors or components that allow MAPCs to retain the ability to differentiate into cell types of more than one embryonic lineage, such as all three lineages. This may be indicated by the expression of specific markers of the undifferentiated state, such as Oct 3/4 (Oct 3A) and/or markers of high expansion capacity, such as telomerase.

### Pharmaceutical Formulations

In certain embodiments, the cell populations are present within a composition adapted for and suitable for delivery, i.e., physiologically compatible.

In some embodiments the purity of the cells for administration with or to the islets is about 100% (substantially homogeneous). In other embodiments it is 95% to 100%. In some embodiments it is 85% to 95%. Particularly, in the case of admixtures with other cells, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the cells have undergone, for example, 10-20, 20-30, 30-40, 40-50 or more cell doublings.

### Dosing

Doses (i.e., the number of cells) for humans or other mammals can be determined without undue experimentation by the skilled artisan, from this disclosure, the documents cited herein, and the knowledge in the art. The optimal dose to be used in accordance with various embodiments of the invention will depend on numerous factors, including the following: the disease being treated and its stage; the species of the donor, their health, gender, age, weight, and metabolic rate; the donor's immunocompetence; other therapies being administered; and expected potential complications from the donor's history or genotype. The parameters may also include: whether the cells are syngeneic, autologous, allogeneic, or xenogeneic; their potency; the site and/or distribution that must be targeted; and such characteristics of the site such as accessibility to cells. Additional parameters include co-administration with other factors (such as growth factors and cytokines). The optimal dose in a given situation also will take into consideration the way in which the cells are formulated, the way they are administered (e.g., perfusion, intra-organ, etc.), and the degree to which the cells will be localized at the target sites following administration.

### Compositions

The invention is also directed to cell populations with specific potencies for achieving any of the effects described herein. As described above, these populations are established by selecting for cells that have desired potency. These populations are used to make other compositions, for example, a cell bank comprising populations with specific desired potencies and pharmaceutical compositions containing a cell population with a specific desired potency.

### Example

C57BL/6 mice were used as islet donors and transplant recipients in all procedures.

### Preparation and Characterization of Human MAPC

### Preparation of MAPC Expansion Medium

DMEM is mixed with MCDB-201 solution at a 60:40 volume:volume ratio.

500 mL base medium is supplemented with the following reagents;
(a) 1 mL of 500× Insulin-Transferrin-Selenium.
(b) 2.5 mL of 100× Linoleic Acid-Bovine Serum Albumin
(c) 5 mL of 10,000 U/mL Penicillin-Streptomycin.
(d) 5 mL of 10⁻⁴ M L-Ascorbic Acid.
(e) 100 μL of 50 μg/mL hPDGF.
(f) 200 μL of 25 μg/mL hEGF.
(g) 100 μL 0.25 mM dexamethasone.
(h) Fetal Bovine Serum to 18%.

### Preparation of Fibronectin Coating Solution

1× fibronectin (100 ng/mL) coating solution is made by diluting 50 μL of 0.1% fibronectin into 500 mL of PBS. The solution can be stored at 4° C.

T75 culture flasks are coated for at least 30 minutes in a 37° C., 5.5% CO₂ incubator with 5 mL coating solution.

### MAPC Isolation From Bone Marrow Aspirate

Fresh marrow can be used but aspirates can also be stored overnight. The aspirate is transferred into a 50 mL centrifuge tube with an equal volume of PBS. 20 mL of this is layered on top of 20 mL Histopaque-1077. This is centrifuged for 1,000×g for 20 minutes at room temperature. The mononuclear layer is collected and diluted to 50 mL with PBS. This is centrifuged at 350×g for 5 minutes. The supernatant is removed and the cells are resuspended in 50 mL of PBS. This is centrifuged at 350×g for 5 minutes. The supernatant is removed and the cells are resuspended in around 1-2 mL of PBS. The cells are seeded at a density of 0.5-1.0×10⁶ cells/cm² in 15 mL of medium on 1× fibronectin-coated T75 flasks. The cells are incubated in 5.5% CO₂, 5% O₂, at 37°. After 24 hours the medium is removed and the cells are rinsed about three times with 5 mL of PBS to removed non-adherent cells. For expansion 10 mL of fresh medium is added. The cells are cultured for 5-8 days. The culture medium is replaced every 2-3 days. Cells undergo clonal expansion and will become visible as distinct cell clusters. When the clonal expansion clusters reach a confluency of 50-70% (within the clusters) the cells are passaged.

### MAPC Subculturing and Expansion

The cells are detached with Trypsin-EDTA solution. The reaction is stopped by adding the collected expansion medium. The cells are centrifuged for 5 minutes at 350×g. The cells are then resuspended in MAPC expansion medium and seeded at a density of 500 cells/cm² in 1× fibronectin coated flask. They are then incubated as above. The cells are passaged every other day to maintain cultures at low density.

The following instructions are noted. Serum may provide a significant source of variability. The optimum serum concentrations can vary depending on serum batch characteristics. Accordingly, different serum lots are screened for their capacity to support optimal MAPC expansion. A large quantity of serum from an appropriate batch can be reserved. Ideally, MAPC are seeded at densities between and 200 and 2,000 cells per cm² and higher densities are avoided. They are constantly passaged at sub-confluency (30-70%). Using these conditions the MAPCs can be routinely expanded for up to 15 to 20 passages (50-70 population doublings).

Human MAPC (n=2) used in this study were isolated from bone marrow as described above. At about 24 cell doublings the cells were frozen as a “cell bank” Then the cells were thawed and expanded until population doubling of about 27 in a Quantum bioreactor as is described in U.S. 2012/0308531, which is incorporated by reference for disclosing methods for expanding MAPCs in the bioreactor. This closed automated culture system is comprised of a synthetic hollow-fiber bioreactor connected to sterile closed-loop, computer-controlled media and gas exchangers. The bioreactor contains ˜11,000 fibers generating an expansion surface area of 2.1 m². After coating the bioreactor with fibronectin, cells were seeded on the inside of the hollow fibers at about 2200/cm² and expanded in MAPC culture medium. Cells were harvested after 5-6 days using trypsin/EDTA (fibronectin coating). Then the harvested cells were expanded in the bioreactor until approximately population doubling 33. They were seeded at about 400/cm². The coating was cryoprecipitate. Those cells (at population doubling 33) were used in the experiments exemplified in this application.

Phenotypic analysis of the human MAPC was performed using fluorochrome-conjugated antibodies recognizing cluster of differentiation 3 (CD3), CD31, CD34, CD40, CD44, CD86, CD105, Flk1, HLA-ABC, and HLA-DR (ebioscience Inc., San Diego, Calif.). Acquisition was done by using a Gallios™ multicolor flow cytometer (Beckman Coulter, Suarlée, Belgium). For analysis of the samples, FlowJo (Tree Star Inc., Ashland, Oreg.) software was used.

Cell-free supernatants were assayed for human basic fibroblast growth factor (bFGF), C-reactive protein (CRP), eotaxin, eotaxin-3, soluble fms-like tyrosine kinase 1 (sFlt1), granulocyte-macrophage colony-stimulating factor (GM-CSF), soluble intracellular adhesion molecule-1 (sICAM-1), interferon-γ (IFN-γ), interleukin-1α (IL1α), IL1β, IL10, IL12 p70, IL12/IL23p40, IL13, IL15, IL16, IL17A, IL2, IL4, IL5, IL6, IL7, IL8, IFN-γ-induced protein-10 (IP-10), monocyte chemoattractant protein-1 (MCP-1), MCP-4, macrophage-derived chemokine (MDC), macrophage inflammatory protein-1α (MIP-1α), MIP-1β, placental growth factor (P1GF), serum amyloid A (SAA), thymus- and activation-regulated chemokine (TARC), Tie2, tumor necrosis factor-α (TNF-α), TNF-β, soluble vascular cell adhesion molecule-1 (sVCAM-1), vascular endothelial growth factor-A (VEGF-A), VEGF-C, and VEGF-D by multiplex electrochemiluminescence (Meso Scale Discovery, Rockville, Md.) as per manufacturer's protocol.

The angiogenic potential of human MAPC was examined in the chick chorioallantoic membrane (CAM) as described [Movahedi B, et al., (2008) Diabetes 57: 2128-2136].

### Marginal Mass Syngeneic Islet Transplantation Diabetes Model

To induce diabetes in recipients, a single intravenous injection of alloxan (90 mg/kg; Sigma-Aldrich) was administered to male C57BL/6 mice, and animals were considered to be diabetic after two consecutive non-fasting tail vein blood glucose concentrations of >200 mg/dl, measured by an AccuCheck Glucometer (Roche Diagnostics Vilvoorde, Belgium). Before transplantation, islets from 2-3 week-old C57BL/6 mice isolated by collagenase digestion were washed, counted, and in some cases mixed with human MAPC [Baeke F, et al., (2012) Diabetologia 55: 2723-2732]. Thereafter, the cellular pellets were transferred to silicon microtubing (Becton Dickinson, Erembodegem, Belgium), centrifuged for 5 minutes at 1500 rpm. During transplantation, the mice were anaesthetized and the left kidney was exposed by a lumbar incision. Diabetic recipient mice were given 150 islets alone, 150 islets and 250,000 human MAPC as separate pellets (SEP) or 150 islets and 250,000 human MAPC as composite pellet (MIX) under the renal capsule. Non-fasting blood glucose levels from the tail vein of each recipient were measured daily during the first week post-transplantation and thereafter three times weekly. Mice were considered cured when having blood glucose levels <200 mg/dL after 3 consecutive measurements. All islet transplantations were performed at random in all experimental groups. On week 2 and 5 after islet transplantation, graft-bearing kidneys were removed and fixed in 4% formaldehyde followed by paraffin embedding or were used for RNA isolation.

### Physiological Studies

Glucose tolerance tests were performed after a 16-hour fast. Mice were injected ip with D-glucose (2 g/kg body weight), and blood glucose levels were measured at the indicated times.

For serum insulin and C-peptide determination, blood was collected from the saphenous vein. Serum was isolated by centrifugation, levels of pancreatic hormones were determined by ultrasensitive enzyme-linked immunosorbent assay (ELISA) kits (Mercodia, Uppsala, Sweden; Merck Millipore, Massachusetts, Mass.).

### Morphometry and Immunohistochemistry

Graft-bearing kidneys were imbedded in paraffin and 6 μM sections were obtained from the total graft area. Insulin (guinea-pig, Dako Belgium nv/sa, Heverlee, Belgium), glucagon (mouse, Sigma, St. Louis, Mo.), somatostatin (rat, Abcam, Cambridge, UK), and endomucin (rat, Santa Cruz Biotechnology Inc., Santa Cruz, Calif.) stainings were used to evaluate beta cell mass and blood vessel density respectively with the aid of the Ventana system (Ventana Medical Systems Inc., Tucson, Ariz.). The endomucin antibody is recommended for detection of endomucin of mouse and not human origin.

For quantification of the beta cell and blood vessel volumes, all images were captured using a Nikon Eclipse TE2000-E microscope using a 40× magnification objective and the large image-capture feature so that the entire graft area of each section could be pictured at once. Insulin+, glucagon+ and somatostatin+ areas as well as endomucin+ areas within the endocrine compartment of the islet graft were measured semi-automatically by ImageJ/Fiji software on approximately 15% (i.e. every fifth section) of the total graft as described [Coppens V, et al. (2013) Diabetologia 56: 382-390]. The vessel/beta cells ratio was calculated as (blood vessel area/insulin+area)×100%. Vessel density was calculated as the number of intra-islet vessels per mm².

### Quantitative PCR

Islet graft RNA was isolated as described [Ding L, et al. (2015) Cell Transplant 24: 1585-1598], and a 1-μg aliquot was reverse transcribed into cDNA (Superscript II; Life Technologies, Carlsbad, Calif.). cDNA was then subjected to quantitative PCR using gene-specific forward and reverse primers using either Fast SYBR® Green Master Mix or a gene-specific TaqMan® probe in combination with TaqMan® Fast Universal Master Mix (Life Technologies). Primer and probes sequences are listed in Supplementary table 1. Each quantitative reaction was carried out in duplicate or triplicate, and islet grafts from 6-11 mice of each experimental group were independently tested. Relative mRNA expression value is calculated using the ΔΔCt method. All samples were normalized to the average of Actine, HPRT and RPL27 as housekeeping genes. Background amounts of each target gene were calculated from the non-grafted kidney. Results are expressed as the mean±SEM.

### Statistics

Statistics were calculated with Prism software 5.0 (GraphPad Software Inc., San Diego, Calif.). The chi-square test was applied to identify the significance of the difference between diabetes reversal rates between different groups. All numerical values were presented as the mean±SEM. Significance was determined using Mann-Whitney U-test or Kruskal-Wallis test, and a value of p<0.05 was considered significant.

### Composite Mixture

150 islets were mixed in 30 μl PBS with MAPCs and this composite was transferred to a silicone microtube, centrifuged for 5 minutes at 1500 rpm, and the pellet was transplanted under the kidney capsule.

### Results

Human MAPC Secrete Angiogenic Growth Factors and have Neo-Angiogenic Potential in the In Vivo CAM Assay

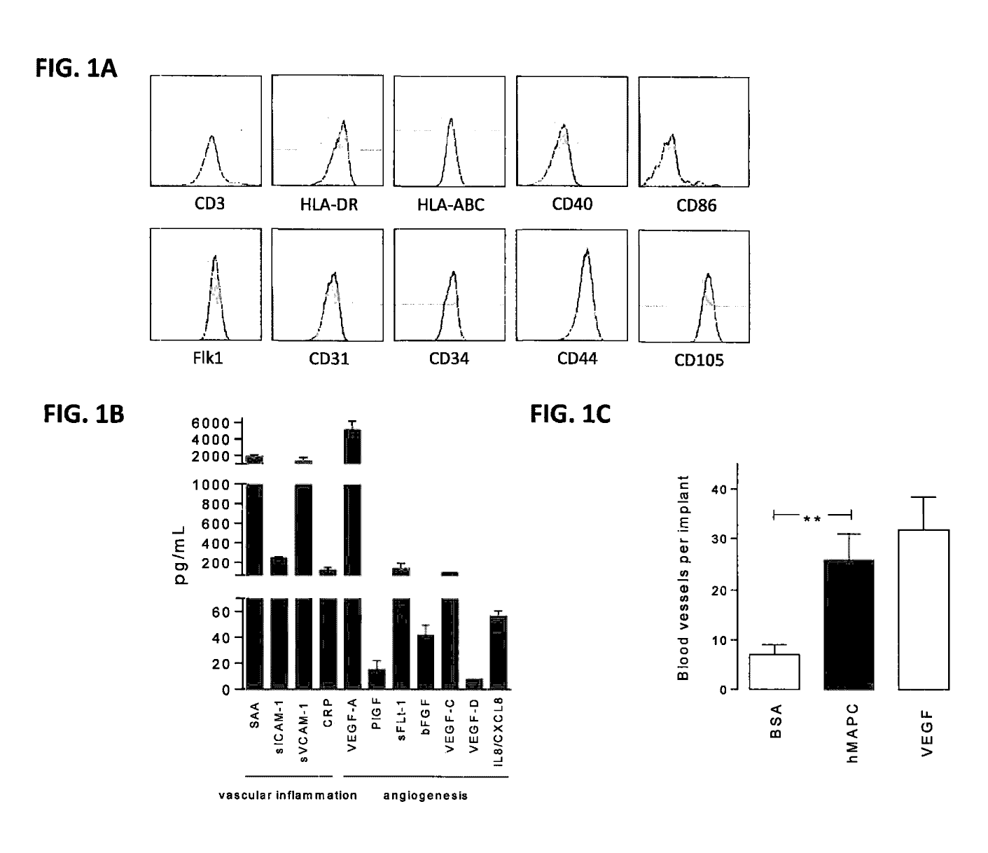

Human MAPC presented a low expression of HLA-ABC (<25%) and lacked expression of HLA-DR, CD40, CD86, CD3, Flk1/VEGFR2/KDR, CD31/PECAM-1, and CD34 (<1%), which are typical cell surface markers for MHC class II and co-stimulation molecules, T cells and endothelial cells, respectively (FIG. 1A). Human MAPC were positive for CD44 and CD105 (>95%) [Reading J L, et al. (2013) J Immunol 190: 4542-4552]. Their surface marker signature defines a unique phenotype that distinguishes them from any other known class of stem cells [Reading J L, et al. (2013) J Immunol 190: 4542-4552].

Culture supernatant of human MAPC was analyzed with human biomarker 40-Plex kit containing a pro-inflammatory panel, cytokine panel, chemokine panel, angiogenesis panel and vascular inflammation panel (FIG. 1B). The cells produced numerous angiogenic growth factors, including VEGF (VEGF-A, -C and -D), P1GF, sFlt-1, bFGF, and IL8. On the other hand, the cells had negligible secretion of various cytokines (i.e. IFN-γ, IL1α, IL1β, IL2, IL4, IL5, IL6, IL7, IL10, IL12p70, IL12/IL23p40, IL13, IL15, IL16, IL17A, TNF-α, and TNF-β), and chemokines (Eotaxin, Eotaxin-3, IP-10, MCP-1, MCP-4, MDC, MIP-1α, MIP-1β, and TARC)(data not shown).

The neo-angiogenic potential of human MAPC was tested using the CAM angiogenesis model. Inoculation with 5 μg recombinant human VEGF markedly increased the number of blood vessels directed toward the implant (FIG. 1C). On day 13, there were approximately 4.5-fold more vessels compared to control implants containing 50 μg BSA. Human MAPC (2.5×10⁵) significantly increased vessel formation by 3.5-fold compared to controls (FIG. 1C).

### Co-Transplantation of Islet-Human MAPC as a Composite Pellet Improves the Outcome of Marginal Mass Islet Transplantation

The number of pancreatic islets transplanted was titrated to determine ‘a marginal islet mass’ that would be just at the edge of achieving normoglycemia in around 50% of recipients. Transplantation of 50 syngeneic C57BL/6 islets did not reverse hyperglycemia (0 out of 7 mice), whereas 100% (4 out of 4 mice) achieved normoglycemia when 300 islets were transplanted under the kidney capsule. We assessed that the marginal islet number was approximately 150 islets (25 out of 45 mice, 56% achieving normal blood glucose concentrations 5 weeks post-transplantation). This number of islets was selected for further experiments.

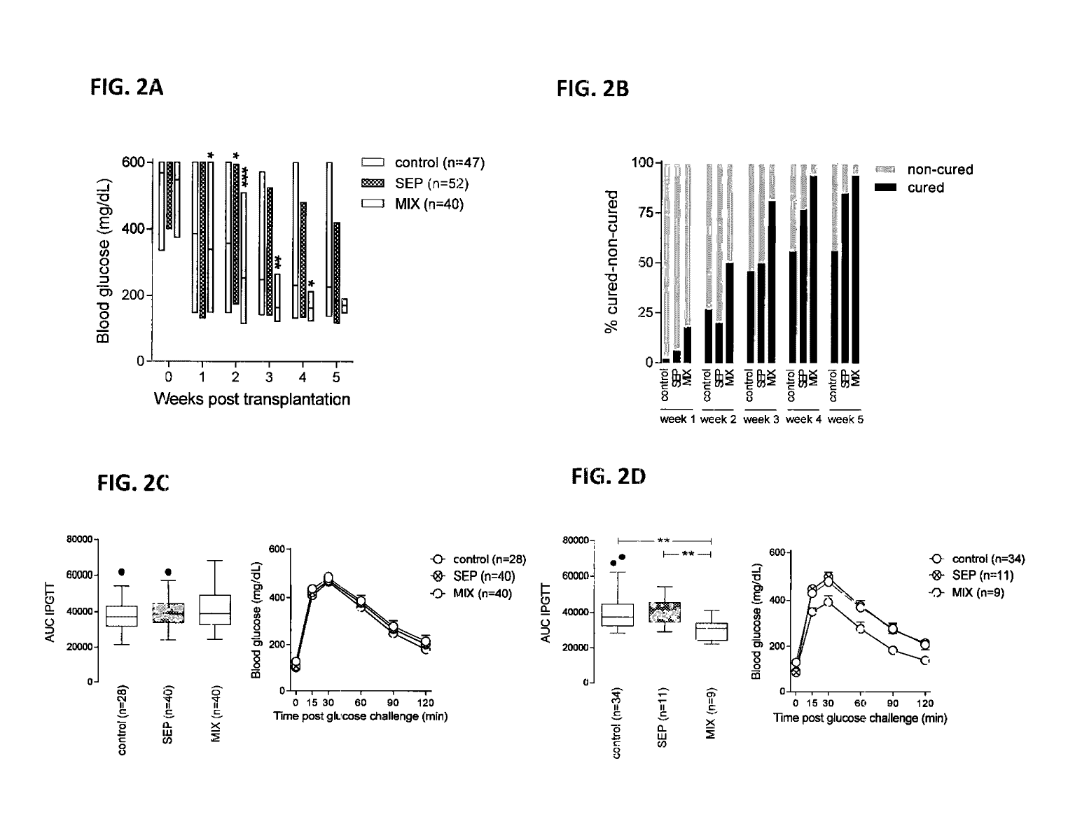

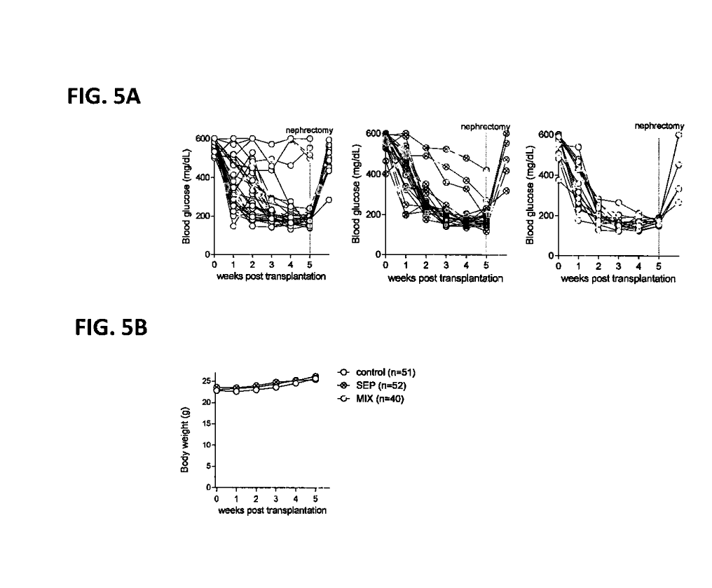

Next, the inventors investigated the outcome of the co-transplanted marginal islet mass with human MAPC as separate or composite pellets and monitored blood glucose levels of transplanted animals up to 5 weeks. Co-transplantation of pancreatic islets with human MAPC as separate pellets (SEP) slightly improved the average blood glucose concentrations compared to mice transplanted with islets alone. Interestingly, mice receiving islet-human MAPC composites (MIX) had better glycemic control at all measured time points from 2 weeks onwards (FIG. 2A). Three weeks after transplantation, 81% of the mice transplanted with islet-human MAPC composites (13 out of 16 mice in the MIX group) were normoglycemic compared to 50% of the mice transplanted with islet-human MAPC as separate pellets (13 out of 26 mice in the SEP group; p<0.05) and 47% in the mice transplanted with islets alone (21 out of 45 mice in the control group; p<0.05)(FIG. 2B). By the end of the observation period (week 5 post-transplantation), even a greater proportion of mice co-transplanted with islet-human MAPC reversed diabetes compared to mice transplanted with islets alone (94% in the MIX group, p<0.01 and 85% in the SEP group, p<0.001 versus 56% in the control group). After nephrectomy, the blood glucose concentrations of normoglycemic islet recipients rapidly progressed to severe hyperglycemia, indicating that the improvement in metabolic glucose control was resulting from the transplanted syngeneic islets and not from the regeneration of remnant islets in the alloxan-treated pancreas of the islet recipients (FIG. 5A). Moreover, there was no significant difference in body weight between transplant recipients from different experimental groups on day 0 (22.8±0.27, 23.5±0.2 and 22.9±0.21 g for the control, SEP, and MIX groups, respectively, n=40-52) or at week 5 post-transplantation (25.6±0.33, 26.2±0.32 and 25.4±0.27 g, for the control, SEP, and MIX groups, respectively, n=40-52) (FIG. 5B).

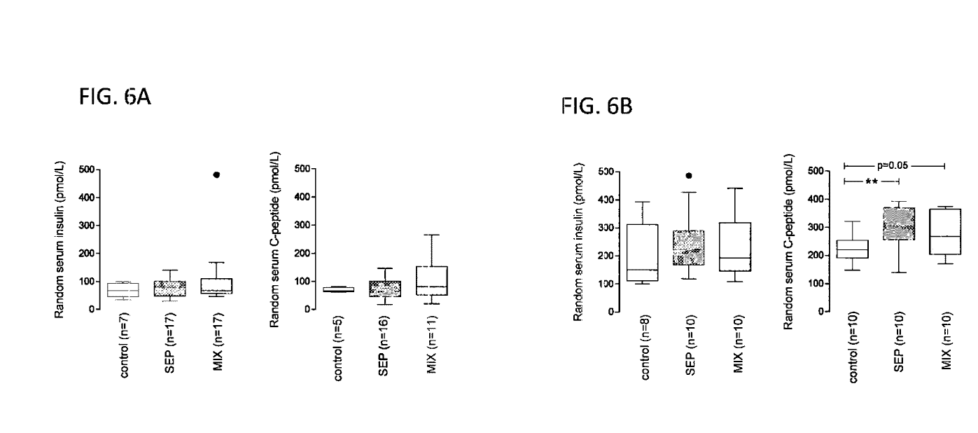

Serum mouse insulin and C-peptide levels were measured 2 and 5 weeks after transplantation, as an index of islet graft function. At week 2 post-transplantation, insulin and C-peptide concentrations were not significantly different between the various experimental groups (FIG. 6A). However, at week 5 post-transplantation, C-peptide values were significantly better in mice co-transplanted with islet-human MAPC as separate pellets (304±80 pmol/1 in the SEP group; n=10, p<0.01) as well as composite pellets (282±77 pmol/1 in the MIX group; n=10, p=0.05) compared to values from the islet-alone mice (232±52 pmol/1 in the control group; n=10) (FIG. 6B).

To investigate the insulin secretory capacity of the islet transplant, a series of intraperitoneal glucose tolerance tests (IP-GTT) were performed week 2 and 5 post-transplantation. At week 2 post-transplantation, there were no significant differences in glucose clearance among the studied groups. On the other hand, at week 5 post-transplantation, mice co-transplanted with islet-human MAPC composites (MIX) cleared glucose more efficiently than mice transplanted with islets and human MAPC as separate pellets (SEP) or with islets alone (control)(FIG. 2C-D). To further support the observations of the IP-GTT, area under the curve (AUC) was calculated and found to be significantly different between the group transplanted with islet-human MAPC composites (MIX) compared to the group transplanted with islets and human MAPC as separate pellets (SEP) (p<0.01) or transplanted with islets alone (control)(p<0.01). (FIG. 2C-D).

Increased Beta- and Alpha-Cell Volume and Blood Vessel Formation in Mice Transplanted with Islet-Human MAPC Composites

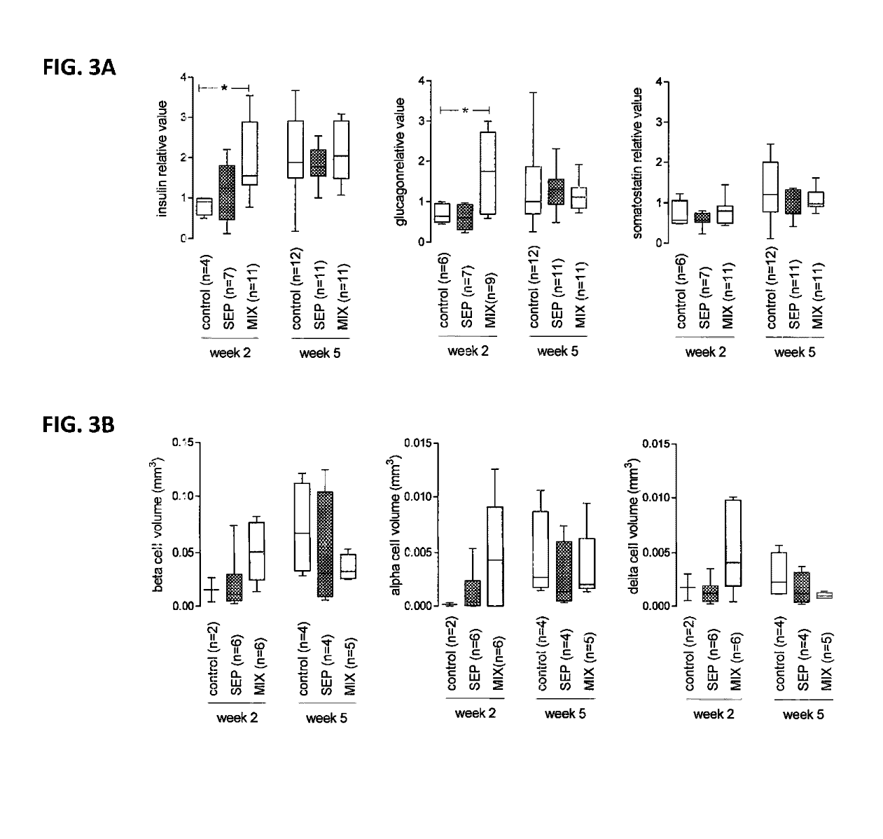

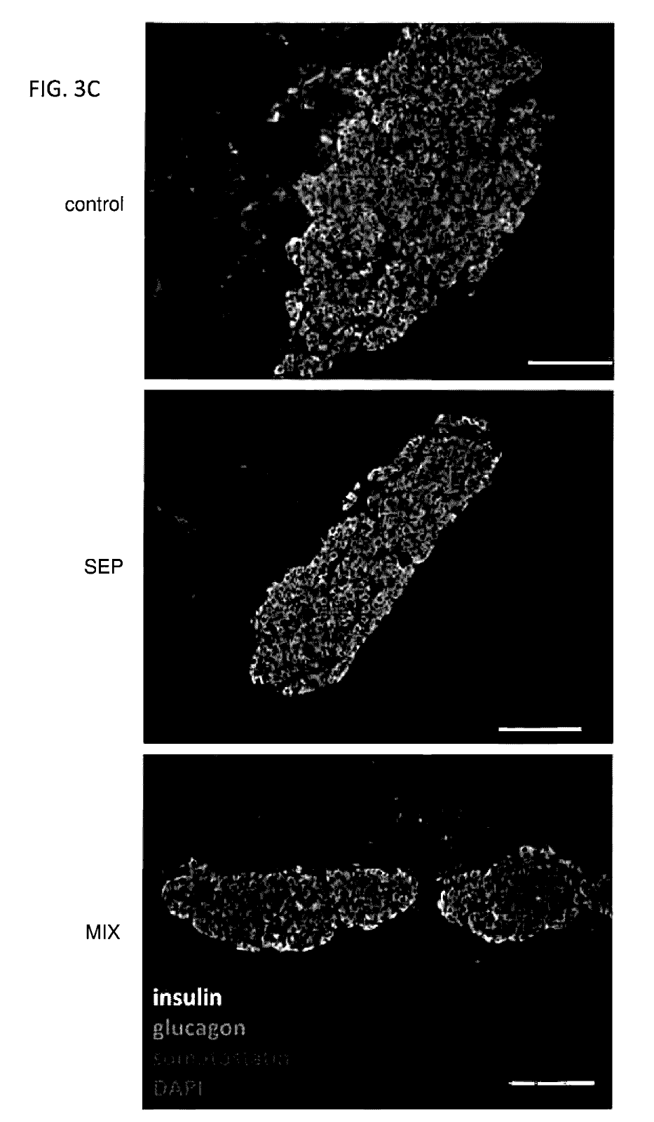

Grafts from the co-transplant and islet-alone groups were evaluated for their gene profile, cytoarchitecture and revascularization process. Insulin and glucagon mRNA expression levels were higher in mice co-transplanted with islet-human MAPC composites (MIX) compared to those of the islet-alone group (control) 2 weeks after transplantation. There was no difference in somatostatin mRNA expression levels at this time point. At week 5 post-transplantation, the intra-graft mRNA levels of the studied endocrine hormones were comparable in all groups. These measures were corroborated by histology of the grafts, showing higher insulin- and glucagon-positive areas in the grafts of mice transplanted with islet-human MAPC composites (MIX), compared to mice transplanted with islets only (control) (FIG. 3B-C).

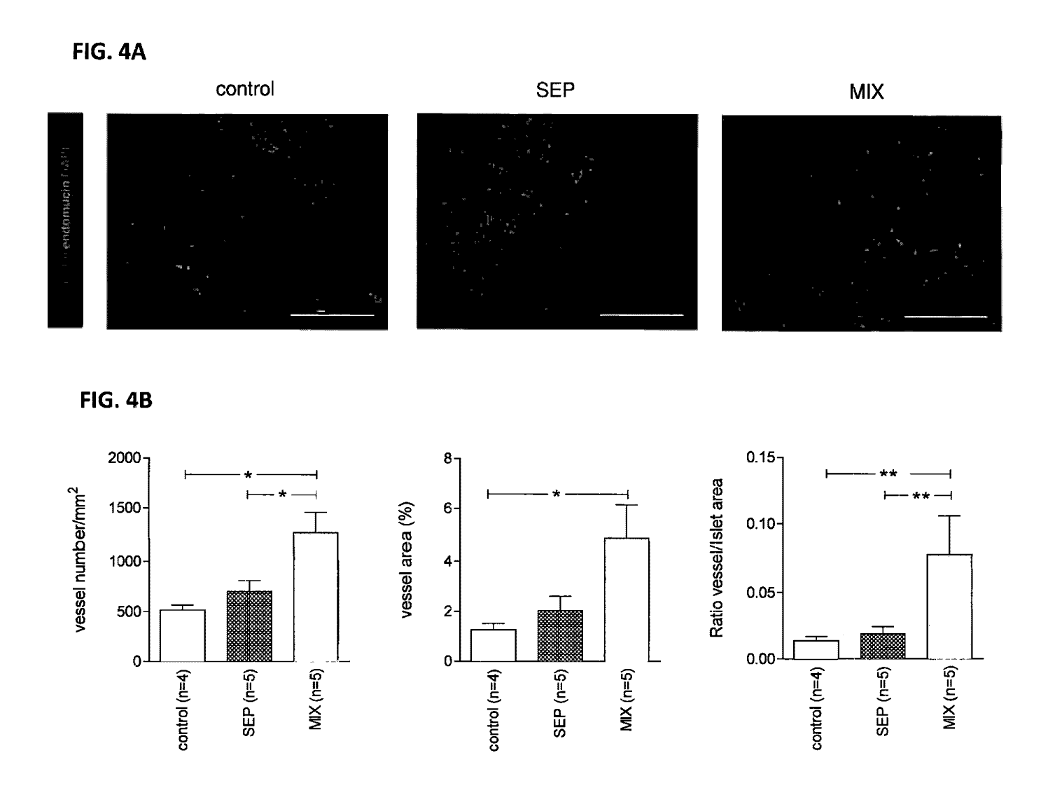

Blood vessel formation was measured by endomucin expression, a marker for vascular endothelial cells. At week 2 post-transplantation, graft vessel density and area as well as ratio of the vessel area over insulin+ area did not differ between the studied groups. At week 5 post-transplantation, enhanced graft revascularization was observed in mice co-transplanted with islets-human MAPC composites (MIX) compared to mice transplanted with islet-human MAPC as separate pellets (SEP) or with islets alone (control). In the MIX grafts, 1256±203 vessels per mm² were detected, compared to 702±106 per mm² in the SEP grafts and 515±52 per mm² in the islet-alone grafts (both p<0.05; FIG. 4B). Another index of neo-angiogenesis, graft vessel area, was significantly higher in the MIX grafts (4.85±1.32%, n=5), than in the SEP grafts (2.04±0.57%, n=5) or the grafts of islets only (1.26±0.25%; n=4, p<0.05, FIG. 4B). Additionally, mice transplanted with the MIX grafts had a higher ratio of vessel per insulin-positive area than the SEP grafts and islet-alone grafts (0.079±0.027 versus 0.019±0.006 and 0.014±0.003 vessels per islet, both p<0.01)(FIG. 4B).

Supplementary table 1
Primer and probe characteristics

| | | PCR | Cq |
|---|---|---|---|
| Target | Sequence | efficiency | range |
| insulin | F 5′-CCGGGAGCAGGTGACCTT-3′ (SEQ ID NO: 1) | 105 | 25-32 |
| | R 5′-GATCTACAATGCCACGCTTCTG-3′ (SEQ ID NO: 2) | | |
| | P 5′-AGACCTTGGCACTGGAGGTGGCC-3′ (SEQ ID NO: 3) | | |
| glucagon | F 5′-AACAACATTGCCAAACGTCA-3′ (SEQ ID NO: 4) | 92 | 21-32 |
| | R 5′-TGGTGCTCATCTCGTCAGAG-3′ (SEQ ID NO: 5) | | |
| | SYBR | | |
| somatostatin | F 5′-GGAAACAGGAACTGGCCAAGT-3′ (SEQ ID NO: 6) | 106 | 25-32 |
| | R 5′-GGGTTCGAGTTGGCAGACC-3′ (SEQ ID NO: 7) | | |
| | SYBR | | |

F: forward; R: reverse; P; probe; SYBR: Fast SYBR ® Green; Cq (quantification cycle, also referred to as Ct (threshold cycle))

What is claimed is:

1. A composition comprising stem cells and pancreatic islets, wherein the stem cells are non-embryonic, non-germ, express telomerase, are not tumorigenic, and can undergo more than 40 doublings in culture.

2. The composition of claim 1 in culture medium.

3. The composition of claim 1 wherein the stem cells and pancreatic islets are admixed with a pharmaceutically-acceptable carrier.

4. The composition of claim 1 wherein the stem cells and islets are in a composite pellet in which the islet cells and stem cells are in direct physical contact.

5. The composite pellet of claim 4 wherein the stem cells adhere to and/or coat the islets.

6. The composition of claim 1 wherein the islets and/or the stem cells have been expanded in culture.

7. The composition of claim 1 wherein the ratio of stem cells to islets is about 2500:1.

8. The composition of claim 1 wherein the amount of stem cells and the amount of islets are sufficient to improve blood glucose levels in a subject with diabetes when administered to the subject.

9. A method for making the composition of claim 1 comprising admixing pancreatic islets and stem cells.

10. The method of claim 9 where the pancreatic islets and stem cells form a composite pellet.

11. The method of claim 10 where the pellet is formed by centrifugation.

12. The method of claim 10 where the pellet is formed by co-culture.

13. The method of claim 12 comprising seeding pancreatic islets and stem cells together in culture.

14. A method to improve pancreatic islet viability ex vivo, the method comprising contacting pancreatic islets with stem cells prior to transplantation to a subject, wherein the stem cells are non-embryonic, non-germ, express telomerase, are not tumorigenic, and can undergo more than 40 doublings in culture.

15. The method of claim 14 wherein the stem cells and islets are co-encapsulated.

16. The method of claim 14 wherein the pancreatic islets and stem cells are contacted ex vivo.

17. The method of claim 16 wherein the ex vivo contact is in cell culture.

18. The method of claim 14 wherein the pancreatic islets and/or the stem cells have been expanded in vitro.

19. The method of claim 14 wherein the pancreatic islets and stem cells are contacted to form a composite pellet.

20. A method to treat diabetes, the method comprising co-administering stem cells and pancreatic islets in an effective amount to treat diabetes, wherein the stem cells are non-embryonic, non-germ, express telomerase, are not tumorigenic, and can undergo more than 40 doublings in culture.

21. The method of claim 20 wherein the stem cells and islets are co-administered in a composite pellet.


SEQUENCE LISTING (7 sequences)

SEQ ID NO: 1 - DNA, 18 bp, Artificial Sequence
  Description of Artificial Sequence Synthetic primer
ccgggagcaggtgacctt

SEQ ID NO: 2 - DNA, 22 bp, Artificial Sequence
  Description of Artificial Sequence Synthetic primer
gatctacaatgccacgcttctg

SEQ ID NO: 3 - DNA, 23 bp, Artificial Sequence
  Description of Artificial Sequence Synthetic probe
agaccttggcactggaggtggcc

SEQ ID NO: 4 - DNA, 20 bp, Artificial Sequence
  Description of Artificial Sequence Synthetic primer
aacaacattgccaaacgtca

SEQ ID NO: 5 - DNA, 20 bp, Artificial Sequence
  Description of Artificial Sequence Synthetic primer
tggtgctcatctcgtcagag

SEQ ID NO: 6 - DNA, 21 bp, Artificial Sequence
  Description of Artificial Sequence Synthetic primer
ggaaacaggaactggccaagt

SEQ ID NO: 7 - DNA, 19 bp, Artificial Sequence
  Description of Artificial Sequence Synthetic primer
gggttcgagttggcagacc